(19) United States
(12) Patent Application Publication
(10) Pub. No.: US 2008/0051417 A1
(43) Pub. Date: Feb. 28, 2008

(54) Uses of selective cyclooxygenase-1 inhibitors

(76) Inventor: Yochai Birnbaum, Houston, TX (US)

Correspondence Address: Benjamin Aaron Adler;ADLER & ASSOCIATES, 8011 Candle Lane, Houston, TX, 77071, US

(21) Appl. No.: 11/879,634

(22) Filed: Jul. 18, 2007

Related U.S. Application Data
(60) Provisional application No. 60/831,627, filed on Jul. 18, 2006.

(51) Int. Cl. A61K 31/497; A61K 31/05; A61K 31/35; A61K 31/506; A61P 9/00; A61K 31/40; A61K 31/415

(52) U.S. Cl. 514/254.02; 514/275; 514/342; 514/406; 514/419; 514/460; 514/733

(57) ABSTRACT

The present invention discloses effect of non-selective COX1 inhibitor such as aspirin, statins, thiazolidinediones or combinations thereof on COX2. Also disclosed herein is a method to avoid the adverse effects that the non-selective COX1 inhibitor may have when administered with statins and/or thiazolidinediones.

### CROSS REFERENCE TO RELATED APPLICATION

This non-provisional application claims benefit of provisional U.S. Ser. No. 60/831,627, filed on Jul. 18, 2006, now abandoned.

### BACKGROUND OF THE INVENTION

1. Field of the Invention

The present invention relates to the fields of biochemistry and pharmacology. More specifically, the present invention discloses uses of selective cyclooxygenase-1 inhibitors in prevention of cardiovascular events in patients receiving statins and/or thiazolidinediones therapy.

2. Description of the Related Art

The role of aspirin (ASA) in primary and secondary prevention of cardiovascular disease is well established (Dalen, 2006; Patrono et al., 2005). Aspirin has been shown to block cyclooxygenase-1 (COX1) and thus, the production of prostaglandins and thromboxanes. In addition to blocking COX-1, aspirin has also been observed to modify cyclooxygenase-2 (COX2) by acetylation at the Ser⁵³⁰ near the active enzyme site. This acetylation of COX2 restricts access of arachidonic acid to the catalytic core, thereby leading to incomplete reaction. This incomplete reaction further results in the production of 15-hydroxyeicosatetraenoic acid (15-R-HETE) rather than PGH₂ (the precursor of all prostaglandins) (Claria and Serhan, 1995; Serhan, 2005; Schneider and Brash, 2000). 15-R-HETE thus produced, in turn is converted by 5-lipoxygenase to 15(R)-epi-lipoxin A₄ (15-epi-LXA4), also called aspirin-triggered lipoxin (ATL) (Claria and Serhan, 1995; Serhan, 2005). 15-epi-LXA4, thus generated serves as a local anti-inflammatory mediator involved in protean and diverse human diseases including airway inflammation and asthma, arthritis, graft versus host disease, and multiple cardiovascular, gastrointestinal periodontal disease and renal disease (Serhan, 2005; Fiorucci et al., 2003; Gilroy, 2005). Nonsteroidal anti-inflammatory agents other than aspirin, and selective COX2 inhibitors were not able to generate 15-epi-LXA4 (Claria and Serhan, 1995). In fact, selective COX2 inhibitors prevent 15-epi-LXA4 generation by aspirin (Fiorucci et al., 2003).

Aspirin alone does not upregulate COX2 expression, unless it causes direct damage such as gastritis (Fiorucci et al., 2003). In fact, aspirin was observed to suppress expression of COX2 (Xu et al., 1999). In animal models where inflammation (peritonitis or pleuritis) was used to induce expression of COX2, high doses of aspirin (125-200 mg/kg) were used to augment 15-epi-LXA4 production (Chiang et al., 1998; Paul-Clark et al., 2004). However, in normal human volunteers, aspirin at low-doses has shown to increase 15-epi-LXA4 (15ELX) production more than at high-dose. It was reported that blood levels of 15ELX were greater with aspirin at 81 mg/d than with 325 mg/d. At 650 mg/d aspirin did not increase 15-epi-LXA4 at all. In contrast, the inhibition of thromboxane production was dose dependent, reflecting dose-dependent inhibition of COX1 (Chiang et al., 2004). Thus, it might be possible that COX2 activity is completely inhibited at higher doses of aspirin, whereas the activity is altered from PGH₂ to 15-R-HETE production at lower doses. It might also be possible that the functional status of COX2 following aspirin administration depended on the ratio between aspirin and COX2 concentrations. Thus, in inflammatory models with robust COX2 induction there was a need for high-doses of aspirin to alter the enzymes, whereas in models without inflammation (i.e., normal volunteers), a low-dose was sufficient and high-dose aspirin blocks COX2 completely.

It has also been shown recently that Pioglitazone (PIO) and atorvastatin (ATV) increase myocardial levels of 15-epi-LXA4 (Birnbaum et al., in press). Both pioglitazone and atorvastatin (Birnbaum et al., 2005; Atar et al., 2006) increase the expression and activity of cytosolic phospholipase A₂ (cPLA₂) and COX2 in the rat heart. Additionally, inducible nitric oxide synthase (iNOS) activates COX2 by S-nitrosylation (Atar et al., 2006; Kim et al., 2005). Although this S-nitrosylation of COX2 occurs on all its 13 cysteine residues, the S-nitrosylation of Cys⁵²⁶ is responsible for COX2 activation, at least as assessed by PGE₂ production (Kim et al., 2005). Atorvastatin activates COX2 by inducing iNOS that S-nitrosylates COX2 (Atar et al., 2006). However, the activation of COX2 by pioglitazone is not clear since it does not augment iNOS expression and calcium-independent nitric oxide synthase activity (Atar et al., 2006). In contrast to aspirin, pioglitazone and atorvastatin (Birnbaum et al., 2005; Atar et al., 2006) increase the production of both PGI₂ (prostacyclin) and PGE₂. It is still unclear how atorvastatin and pioglitazone alter COX2 activity to produce 15-R-HETE in addition to PGH₂ (the precursor of prostaglandins).

Currently there are no selective COX1 inhibitors for clinical use. Pharmaceutical companies have concentrated on developing selective COX2 inhibitors, based on the hypothesis that COX2 is responsible for inflammation and development of cancer. However, clinical trials with COX2 inhibitors showed that these inhibitors increased the risk of cardiovascular events, mainly by promoting thrombosis. Since aspirin is known to be useful in the treatment of atherosclerosis, it is currently being used in combination with statins and/or thiazolidinediones.

Despite their combined use, the prior art lacks knowledge of the effects of administration of aspirin, statins, thiazolidinediones or combination thereof. The present invention fulfills this long-standing need and desire in the art.

### SUMMARY OF THE INVENTION

In one embodiment of the present invention, there is provided a method of preventing cardiovascular events in an individual. Such a method comprises administering a pharmacologically effective amount of a composition comprising a non-aspirin selective COX1 inhibitor to the individual and administering pharmacologically effective amounts of statins, thiazolidinediones or a combination thereof, thereby preventing cardiovascular events in the individual.

In another embodiment of the present invention, there is provided a method of preventing formation of blood clots in an individual receiving statin and/or thiazolidinedione. Such a method comprises administering a pharmacologically effective amount of a composition comprising a non-aspirin selective COX1 inhibitor to the individual such that the non-aspirin selective COX1 inhibitor reduces the platelet activity and thereby prevents the formation of blood clots in the individual receiving statin and/or thiazolidinedione.

In yet another embodiment of the present invention, there is provided a method of treating an individual at risk for or with an established cardiovascular disease. Such a method comprises administering pharmacologically effective amounts of a non-aspirin selective COX-1 inhibitor and administering pharmacologically effective amounts of atorvastatin, pioglitazone or a combination thereof, thereby treating the individual at risk for or with the established cardiovascular disease.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows treatment protocols. * oral PIO (10 mg/kg/d) amd ATV (10 mg/kg/d).

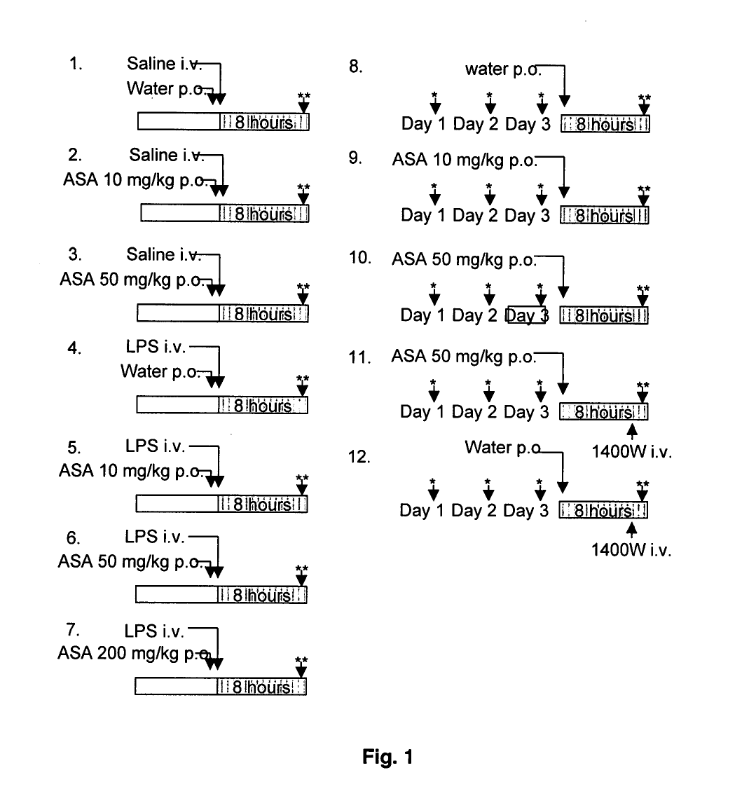

FIGS. 2A-2D shows the effect of LPS and aspirin on myocardial levels of 15-epi-LPXA4, 6-keto-PGF₁_{_{α}} and PGE₂: FIG. 2A shows the effect on 15-epi-LPXA4, FIG. 2B shows the effect on Lipoxin A4, FIG. 2C shows the effect on 6-keto-PGF₁_{_{α}} and FIG. 2D shows the effect on PGE₂. Overall, there were significant differences among groups (p<0.001 for 15-epi-LPXA4, 6-keto-PGF₁_{_{α}} and PGE₂). *—p<0.001 versus the control group; #—p<0.001 versus LPS+aspirin 200 mg/kg; †—p<0.01 versus the control group; ‡—p=0.002 versus LPS+aspirin 200 mg/kg.

FIGS. 3A-3D show the effect of pioglitazone+atorvastatin alone or with aspirin and 1400 W on myocardial levels of 15-epi-LPXA4, 6-keto-PGF₁_{_{α}} and PGE₂. FIG. 3A shows the effect on 15-epi-LPXA4, FIG. 3B shows the effect on Lipoxin A4, FIG. 3C shows the effect on 6-keto-PGF₁_{_{α}} and FIG. 3D shows the effect on PGE₂. Overall, there were significant differences among groups (p<0.001 for 15-epi-LPXA4, 6-keto-PGF₁_{_{α}}, and PGE₂). *—p<0.001 versus the control group; #—p<0.001 versus the pioglitazone+atorvastatin group; †—p<0.02 versus the control group.

FIGS. 4A-4C show the effect of pioglitazone+atorvastatin or lipopolysaccharide on COX2. FIG. 4A shows results of the biotin switch assay that shows S-nitrosylation of COX2 in both the pioglitazone+atorvastatin treated rats (n=4) and lipopolysaccharide-treated rats (n=4). Signal intensity was stronger in the pioglitazone+atorvastatin group than in the lipopolysaccharide group. FIG. 4B shows the results of immunoblot assay. Immunoblotting with COX2 after stripping the membranes showed that the precipitate in both the pioglitazone+atorvastatin and lipopolysaccharide group contained COX2. Signal intensity in the lipopolysaccharide group was stronger than in the pioglitazone+atorvastatin group suggesting greater induction of COX2 by lipopolysaccharide than by pioglitazone+atorvastatin. FIG. 4C shows the ratio of biotinylated COX2 to total COX2 signal density was higher in the pioglitazone+atorvastatin group than in the lipopolysaccharide group.

FIG. 5 shows the effects of acetylation of COX1 and COX2 by aspirin and S-nitrosylation of COX2 by pioglitazone+atorvastatin on the production of 15-R-HETE (the precursor of 15 epi-LPX4) and PGH2 (the precursor of prostaglandins). Acetylation of COX1 led to the inhibition of the enzyme whereas acetylation of COX2 led to the augmentation of 15-R-HETE production and inhibition of PGH2 production. In contrast, S-nitrosylation of COX2 led to augmented production of both PGH2 and 15-R-HETE. However, when COX2 was both nitrosylated and acetylated, the enzyme was inactivated and no 15-R-HETE or PGH2 was produced.

### DETAILED DESCRIPTION OF THE INVENTION

As used herein, the term “a” or “an”, when used in conjunction with the term “comprising” in the claims and/or the specification, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

Aspirin (10-500 mg) is recommended for patients with suspected heart attack by both the American Society of Cardiology/American Heart Association guidelines and the European Society of Cardiology guidelines. A large portion of patients with coronary artery disease or those at risk for developing an artery disease and diabetic patients also receive statins and thiazolidinediones. Hence, some of these patients may receive aspirin, statins and/or thiazolidinediones. Although aspirin has been shown to reduce mortality and morbidity in patients with heart attack, all of these studies were conducted before the widespread use of statins and thiazolidinediones. Thus, the effect of combined administration of aspirin, statins and thiazolidinediones in such individuals is not known.

The present invention discloses the effect of administering aspirin in combination with pioglitazone (PIO) and/or atorvastatin (ATV). In doing so, the present invention examined the effect of different doses of aspirin on myocardial production of 15-epi-LXA4, lipoxin A4, PGE2 and PGI2 by COX2 using two models. One model used lipopolysaccharide to induce COX2 expression whereas the other model used pioglitazone (PIO) and atorvastatin (ATV) to induce COX2 expression.

It was observed that aspirin caused a dose-dependent increase in myocardial 15-epi-LPXA4 levels in rats that were pretreated with LPS, whereas aspirin inhibited the induction of 15-epi-LPXA4 in rats that were pretreated with pioglitazone+atorvastatin. On the other hand, aspirin dose-dependently inhibited the increase in myocardial 6-keto-PGF₁_{_{α}} and PGE₂ in both the models. 15-epi-LPXA4 is known to be produced from 15-R-HETE by 5-lipoxygenase. Until recently it has been reported that 15-R-HETE is produced by COX2 that is acetylated by aspirin at Ser⁵³⁰ (Claria and Serhan, 1995; Serhan, 2005; Schneider and Brash, 2000).

Another potential source of 15-R-HETE is cytochrome P-450 (Claria and Planaguma, 2005; Titos et al., 1999). This pathway has been described in the liver (Claria and Planaguma, 2005; Titos et al., 1999), as well as in human lung adenocarcinoma cell line (A549) (Claria et al., 1996), but not in rat epidermal microsomes (Van Wauwe et al., 1991). In contrast to acetylated COX2 that only produces 15-R-HETE, cytochrome P-450 produces both 15-R-HETE (that is converted to 15-epi-LPXA4 by 5-lipoxygenase) and 15-S-HETE (that is converted to lipoxin A4 by 5-lipoxygenase)(Claria and Planaguma, 2005). Thus, if produced via cytochrome P-450, an equal increase in both lipoxin A₄ and 15-epi-LPXA4 is expected.

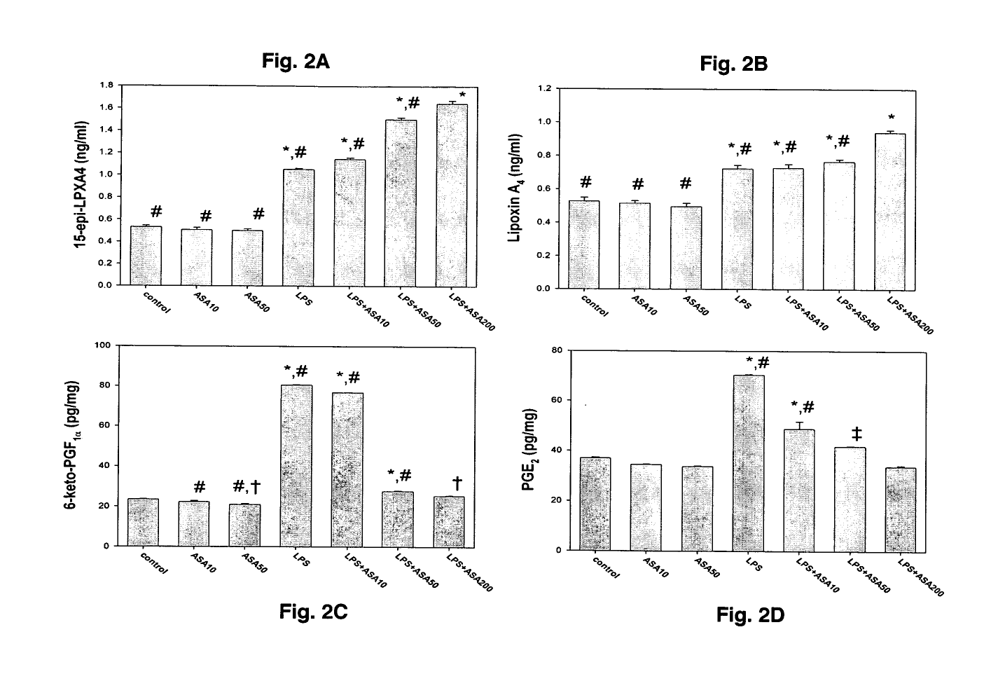

Interestingly, an increase in both lipoxin A₄ and 15-epi-LPXA4 (FIG. 2) was observed in rats treated with LPS. This effect was augmented by aspirin at 200 mg/kg, but not at 10 mg/kg and 50 mg/kg. This finding may favor an involvement of cytochrome P-450 in 15-R-HETE production, at least when high-dose aspirin is added to LPS. However, augmentation of 15-epi-LXA4 production by aspirin (125 mg/kg) was not prevented by a cytochrome P-450 inhibitor in a previous study that used the LPS-induced peritonitis model. This observation suggests that cytochrome P-450 does not significantly contribute to formation of 15-epi-LXA4 in that model (Chiang et al., 1998). Thus, it is possible that the LPS-induction of lipoxin A₄ production is mediated by an interaction between 5-lipoxygenase with 15-lipoxygenase and 12-lipoxygenase (Serhan, 2005; Serhan and Chiang, 2004).

In contrast to the lipopolysaccharide model, rats pretreated with pioglitazone plus atorvastatin demonstrated an increase in 15-epi-LPXA4 without a concomitant increase in lipoxin A₄. This suggested that 15-R-HETE was generated by COX2 and not cytochrome P-450 in this model. It has been previously shown that both pioglitazone and atorvastatin increased myocardial 15-epi-LPXA4 levels, without a significant effect on lipoxin A₄ levels. This dose-dependent augmentation was seen after 3-days of treatment with oral 5 and 10 mg/kg/d, but not with 2 mg/kg/d. Myocardial 15-epi-LPXA4 levels were significantly higher in rats treated with pioglitazone (10 mg/kg/d)+atorvastatin (10 mg/kg/d) than in rats treated with each drug alone. Both atorvastatin and pioglitazone augmented the expression and activity of COX2. The effect of pioglitazone+atorvastatin on myocardial 15-epi-LPXA4 levels was blocked by valdecoxib (a specific COX2 inhibitor) and zileuton (a specific 5-lipoxygenase inhibitor) supporting the production of 15-epi-LPXA4 by the COX2-5-lipoxygenase pathway.

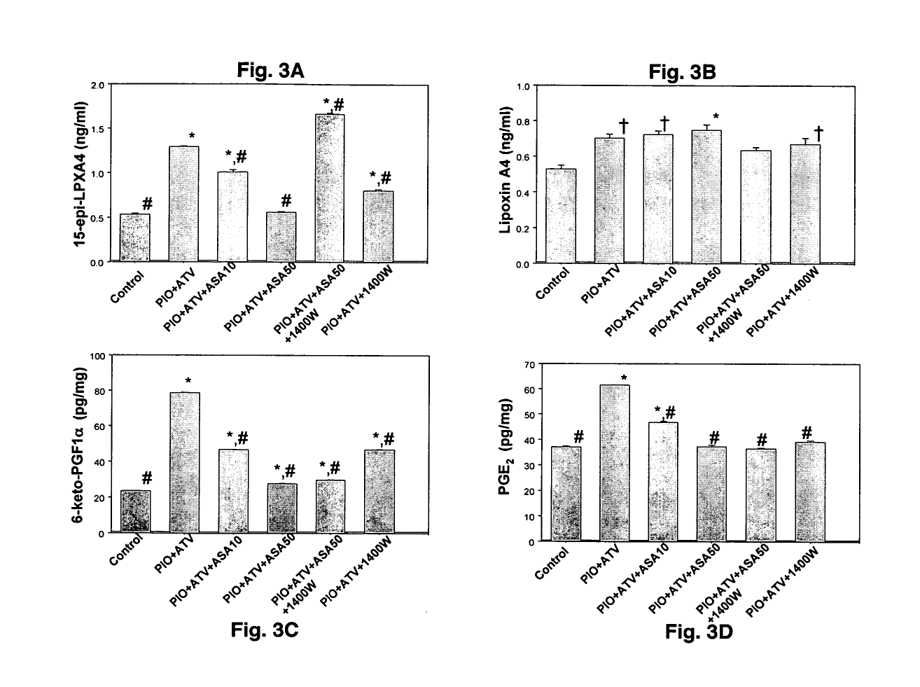

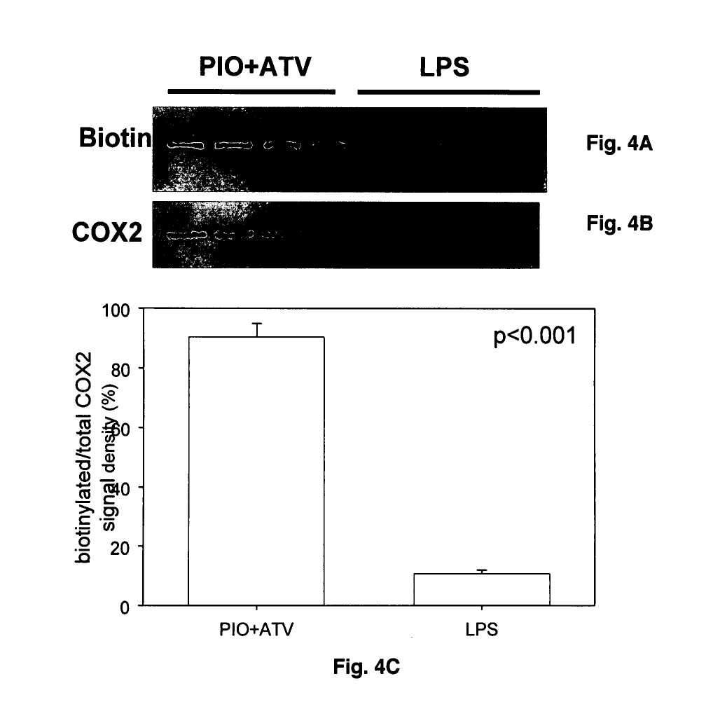

The different effect of aspirin on COX2 induced by pioglitazone+atorvastatin may be explained by the percentage of S-nitrosylation of the enzyme. The “biotin switch” assay clearly showed that a greater percentage of COX2 was S-nitrosylated in rats treated with pioglitazone+atorvastatin than in the lipopolysaccharide treated rats (FIG. 4). Additionally, it has been previously shown that atorvastatin activates COX2 via iNOS-mediated S-nitrosylation (Atar et al., 2006). Intravenous administration of 1400 W at the same dose as used herein, blocked the augmentation of COX2 activity by 3-day pretreatment with atorvastatin (10 mg/kg/d). Using the “Biotin Switch” assay it was demonstrated that the atorvastatin-mediated S-nitrosylation of COX2 was no longer apparent 15 minutes after 1400 W administration, thereby suggesting that S-nitrosylation was reversible. In the present invention, 1400 W blocked the induction of 15-epi-LPXA4 by pioglitazone and atorvastatin, thereby indicating that S-nitrosylation of COX2 was needed for 15-epi-LPXA4 production. Surprisingly, in contrast to the inflammatory model, where aspirin augmented 15-epi-LPXA4 production by LPS (FIG. 2A), aspirin blocked the production of 15-epi-LPXA4 in rats pretreated with pioglitazone+atorvastatin, (FIG. 3A).

The activation of COX2 (at least for the production of PGE₂) by S-nitrosylation occurs at Cys⁵²⁶ (Kim et al., 2005). This site is in close proximity to the site where aspirin acetylates COX2 (Ser⁵³⁰) (Claria and Serhan, 1995; Serhan, 2005; Schneider and Brash, 2000). It was hypothesized that when COX2 is both S-nitrosylated and acetylated, the channel within the enzyme is blocked and COX2 is inactivated. Although both aspirin 50 mg/kg and 1400 W blocked the effect of pioglitazone+atorvastatin when given separately, administration of 1400 W to rats pretreated with pioglitazone+atorvastatin and aspirin 50 mg/kg caused a significant increase in myocardial levels of 15-epi-LPXA4 which was higher than the level in the pioglitazone+atorvastatin alone group (FIG. 3A). This increase could be due to removal of the S-nitrosyl group from Cys⁵²⁶, which reactivated COX2 to produce 15-epi-LPXA4. The full effect of aspirin could be seen since the COX2 molecules were not nitrosylated.

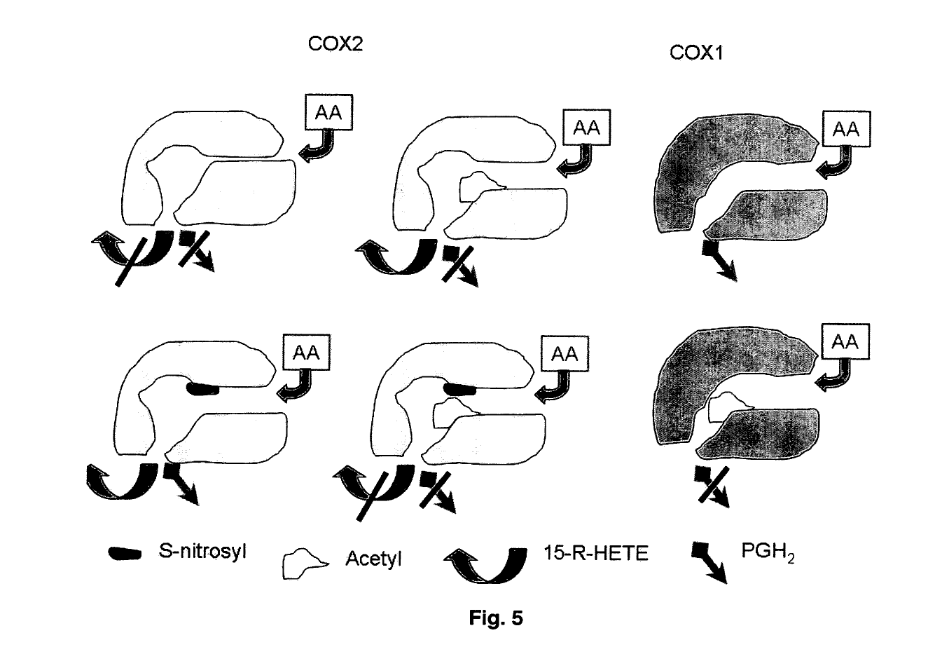

Furthermore, it was observed that COX2 was inactive when it was neither acetylated nor nitrosylated (FIG. 5). Hence, the myocardial levels of PGE₂ and 15-epi-LPXA4 in the rats treated with pioglitazone+atorvastatin and 1400 W without aspirin, were comparable to the levels in the control group (FIG. 3). Interestingly, in this group myocardial 6-keto-PGF₁_{_{α}} level was higher than in the controls. It was previously shown that atorvastatin upregulated PGI₂ synthase expression and activity (Birnbaum et al., 2005). Thus, it was possible that PGI₂ was produced, at least in part by an interaction between COX1 and PGI₂ synthase. When aspirin was added to COX2 that was already S-nitrosylated (by pioglitazone+atorvastatin), there was decrease in the production of both PGH₂ (and therefore, PGI₂ and PGE₂) and 15-epi-LPXA4, thereby supporting a hypothesis that both acetylation and nitrosylation blocked the enzyme completely. When COX2 was only acetylated, without nitrosylation, the enzyme produced 15-R-HETE and therefore 15-epi-LPXA4, but not PGH₂ and prostaglandins (FIG. 5). Therefore, when aspirin was administered to mice pretreated with lipopolysaccharide, acetylation of the non S-nitrosylated COX2 molecules activated them to produce 15-epi-LPXA4. In distinct contrast, when most of the COX2 was S-nitrosylated, as occurred in the pioglitazone+atorvastatin treated animals, the net effect of aspirin was to completely block the activity of COX2.

In general, the findings presented herein disclose complex interactions on activation/inactivation pattern of COX2. Specifically, the findings presented herein suggest a potential adverse interaction between statins, thiazolidinediones and high-dose aspirin. The induction of 15ELX in normal human volunteers was observed to be greater with aspirin at 81 mg/d than with 325 mg/d. At 650 mg/d aspirin did not increase 15ELX at all (Chiang et al., 2004). Although there is no data herein that shows augmentation of 15-epi-LPXA4 production by thiazolidinediones and statins in doses used in the clinical setting, previous studies have shown that atorvastatin blood levels 16 hours after the third oral dose of atorvastatin 10 mg/kg/d (as was used in the present invention) was comparable to that seen in humans treated with atorvastatin 80 mg/d. Thus, it is possible that such interactions concerning the anti-inflammatory and anti-atherosclerosis effects of these drugs may exist in the clinical setting. In humans the anti-platelet effects of aspirin, the lipid-lowering and other pleiotropic effects of statins, and the insulin sensitizing and PPAR-γ activating effects of thiazolidinediones may surpass the interactions on COX2 activity and 15-epi-LPXA4 and prostaglandins production.

In summary, the present invention discloses the risk associated in administering aspirin along with stains and/or thiazolidinediones. Aspirin is administered to patients especially ones at risk or suspected of heart attack. This is because aspirin is known to reduce platelet activity and prevent formation of blood clots by blocking COX1. However, a large number of patients with coronary heart disease or those at risk for developing coronary artery disease or diabetes receive statins and/or thiazolidinediones. Furthermore, it is well-established that statins protect the heart against ischemia-reperfusion injury and that pretreatment with statins reduces the size of experimental myocardial infarction. There are compelling data from both retrospective and prospective clinical studies that show that statins reduce ischemic complications when given to patients before cardiac surgery, major non-cardiac surgery and percutaneous coronary interventions. Furthermore, it is known that COX2 inhibitors completely abrogate the protective effect of statins. Thus, it was necessary to investigate the effect of administering aspirin along with statins and thiazolidinediones.

The results described herein demonstrate that aspirin adversely interacts with statins and thiazolidinediones to reduce their direct protective effect and their anti-inflammatory properties in the long term. It was observed that both atorvastatin and pioglitazone when administered to rats increased myocardial levels of 15-epi-lipoxin A4. Similarly, aspirin when administered alone augmented 15-epi-lipoxin A4 production in rats receiving the pro-inflammatory agent, lipopolysaccharide. In distinct contrast, combined administration of atorvastatin, pioglitazone and aspirin led to decreased production of 15-epi-lipoxin A4. However, addition of a selective iNOS inhibitor to the above mentioned combined administration resulted in increased myocardial 15-epi-lipoxin A4 levels. This is because the iNOS inhibitor removed the nitrosyl group, thus the acetylated COX2 without the nitrosyl group was reactivated to produce 15-epi-lipoxin A4. Thus, there is a differential effect of aspirin, statins and thiazolidinediones on COX2.

These results could be explained as follows: It is known that aspirin acetylates COX-2 and activates the enzyme, thereby producing 15R-HETE, which further generates 15-epi-lipoxins that have anti-inflammatory properties. Statins and thiazolidinediones, by themselves nitrosylate COX2, which also activates the enzyme and produces 15R-HETE, which further generates 15-epi-lipoxins. However, when aspirin is administered with statins and/or thiazolidinediones, COX2 is acetylated and nitrosylated, which causes inactivation of the enzyme and inhibits the production of 15-epi-lipoxins. Thus, aspirin may block some of the favorable non-lipid lowering effects such as direct tissue protection and anti-inflammatory properties of statins and thiazolidinediones.

Furthermore, it was observed that intravenous administration of aspirin (20 mg/kg) at the end of a 30 minute coronary artery occlusion completely abrogated the infarct size limiting the effect of 3-day pretreatment with atorvastatin. This effect was not observed when aspirin was administered alone. Thus, use of non-selective COX inhibitors may increase the risk of cardiovascular events in humans similar to the risk associated with selective COX2 inhibitors. Based on this, it is contemplated that inhibition of thromboxane production by non-aspirin selective COX1 inhibitors may give better results in patients at risk of developing or with established cardiovascular disease that are currently using aspirin, statins and/or thiazolidinediones. Representative selective COX1 inhibitors may comprise of drugs that imitate the effect of aspirin on COX1 without adversely affecting COX2 that can then be used as the preferred anti-platelet therapy for patients receiving statins and/or thiazolidinediones. Examples of selective COX1 inhibitors may not be limited to but comprise SC560, FR122047, Resveratrol, or Valeroyl Salicylate.

The present invention is directed to a method of preventing cardiovascular events in an individual, comprising: administering a pharmacologically effective amount of a composition comprising a non-aspirin selective COX1 inhibitor to the individual, and administering pharmacologically effective amount of statin, thiazolidinedione or a combination thereof, thereby preventing the cardiovascular events in the individual. Generally, the statin, the thiazoldinedione or the combination thereof is administered concurrent with, subsequent to or sequential to the administration of the non-aspirin selective COX1 inhibitor. The non-aspirin selective COX1 inhibitor used herein may inhibit the COX1 activity but may not have any effect on the COX2 activity. Examples of the non-aspirin selective COX1 inhibitor may include but is not limited to SC560, FR122047, Resveratrol, or Valeroyl Salicylate. Further, the statin, the thiazolidinedione or the combination thereof may activate COX2 via nitrosylation. Examples of the statin used herein may include but is not limited to atorvastatin, simvastatin, pravastatin, rosuvastatin, lovastatin, or fluvastatin. Examples of the thiazolidinedione used herein may include but is not limited to pioglitazone, or rosiglitazone.

Furthermore, examples of the individual who may benefit from this method may include but is not limited to those at risk of developing cardiovascular disease, those having an established cardiovascular disease, those going to be subjected to cardiac surgery, non-cardiac surgery, percutaneous coronary interventions or a diabetic.

The present invention is also directed to a method of preventing formation of blood clots in an individual receiving statin and/or thiazolidinedione, comprising the step of administering a pharmacologically effective amounts of a composition comprising a non-aspirin selective COX1 inhibitor to the individual. The administration of the non-aspirin selective COX1 inhibitor may reduce the platelet activity and thereby prevent the formation of the blood clots in the individual receiving statin and/or thiazolidinedione. All other aspects regarding the effect of the non-aspirin selective COX1 inhibitor on COX2, the manner of statin and/or thiazoldinedione administration, effect of the statin and/or thiazolidinedione on COX2, examples of the non-aspirin selective COX1 inhibitor, the statin and thiazolidinedione and the individual benefiting from this method is the same as described supra.

The present invention is further directed to a method of treating an individual at risk for or with an established cardiovascular disease, comprising: administering pharmacologically effective amounts of non-aspirin selective COX1 inhibitor; and administering pharmacologically effective amounts of atorvastatin, pioglitazone or a combination thereof, thereby treating the individual at risk for or with the established cardiovascular disease. Generally, the atorvastatin, pioglitazone or the combination thereof may be administered concurrent with, subsequent to or sequential to the administration of the non-aspirin selective COX1 inhibitor. Furthermore, the non-aspirin selective COX1 inhibitor may block the COX1 activity but may not interfere with the inhibition of COX2 activity by the atorvastatin, the pioglitazone or the combination thereof.

The drugs described herein may be administered independently one or more times to achieve, maintain or improve upon a therapeutic effect. It is well within the skill of an artisan to determine dosage or whether a suitable dosage of the composition comprises a single administered dose or multiple administered doses. An appropriate dosage depends on the subject's health, attainment of the required effect of the drug (for instance, prevention of formation of blood clots, prevention of inflammation, etc) and the formulation used.

The following examples are given for the purpose of illustrating various embodiments of the invention and are not meant to limit the present invention in any fashion.

### EXAMPLE 1

### Rats

Male Sprague-Dawley rats were used in the experiments discussed herein. All the animals received humane care in compliance with “The Guide for the Care and Use of Laboratory Animals” published by the US National Institutes of Health (NIH Publication No. 85-23, revised 1996).

### EXAMPLE 2

### Materials

Pioglitazone was provided by Takeda Pharmaceuticals North America, Inc. (Lincolnshire, Ill.). Crushed tablets for atorvastatin (Pfizer Pharmaceuticals, New York, N.Y.) were used. Aspirin and 1400 W were purchased from Sigma (St Louis, Mo.). ELISA kits for 15-epi-LPXA4 and lipoxin A were purchased from Oxford Biomedical Research (Oxford, Mich.) and for 6-keto-PGF₁_{_{α}} and PGE₂ from Cayman Chemicals (Ann Arbor, Mich.). Polyclonal anti-COX2 antibodies were purchased from Cayman chemicals, N-(3-maleimidopropionyl) biocytin (MPB) from Molecular Probes (Eugene, Oreg.) and immunopure horseradish peroxidase-conjugated streptavidin from Pierce Biotechnology (Rockford, Ill.).

### EXAMPLE 3

### Drugs and Pretreatment

Rats received the following treatment (4 rats in each group): 1) i.v. saline and oral water; 2) i.v. saline and oral aspirin 10 mg/kg; 3) i.v. saline and oral aspirin 50 mg/kg; 4) i.v. LPS and oral water; 5) i.v. LPS and oral aspirin 10 mg/kg; 6) i.v. LPS and oral aspirin 50 mg/kg; 7) i.v. LPS and oral aspirin 200 mg/kg; 8) oral pioglitazone (10 mg/kg/d) and atorvastatin (10 mg/kg/d); 9) oral pioglitazone+atorvastatin and aspirin 10 mg/kg; 10) oral pioglitazone+atorvastatin and aspirin 50 mg/kg; 11) oral pioglitazone+atorvastatin and aspirin 50 mg/kg and i.v. 1400 W, a specific iNOS inhibitor; or 12) oral pioglitazone+atorvastatin, oral water, and i.v. 1400 W, a specific iNOS inhibitor. LPS (10 mg/kg) was dissolved in 1 ml of saline and was administered intravenously 8 hours before harvesting the hearts. Aspirin was dissolved in water and administered by oral gavage 8 hours before harvesting the hearts. Atorvastatin and pioglitazone were administered by oral gavage once daily for 3 days; last dose was administered 16 hours before harvesting the hearts. 1400 W (1 mg/kg) was administered i.v. 15 minutes before harvesting the hearts. All hearts were analyzed for myocardial 15-epi-LPXA4, lipoxin A₄, 6-keto-PGF₁_{_{α}} (the stable metabolite of PGI₂) and PGE₂ levels.

### EXAMPLE 4

### Elisa

The hearts were rapidly explanted, rinsed in cold PBS (pH 7.4), containing 0.16 mg/ml heparin to remove red blood cells and clots, frozen in liquid nitrogen and stored at −70° C. For 15-epi-LPXA4 and lipoxin A₄ determination, myocardial samples from the anterior left ventricular wall were homogenized in ethanol (5 ml/g) and centrifuged at 10,000 g X15 min at 4° C. The supernatant was diluted with water and acidified to pH 3.5 with 1N HCl. The sample was loaded into C-18 Sep-Pak light column (Waters Corporation, Milford, Mass.) and washed with 1 ml of water followed by 1 ml of petroleum ether. The sample was eluted with 2 ml of methyl formate. The methyl formate was evaporated with N₂ and the residue was dissolved in extraction buffer. The manufacturer instruction of the ELISA Kits was followed. For myocardial 6-keto-PGF₁_{_{α}} and PGE₂ levels myocardial samples were homogenized in RIPA lysis buffer with 10% proteinase inhibitor (Sigma) and centrifuged. The supernatants were collected and stored on ice. Measurement of 6-keto-PGF₁_{_{α}} (the stable hydrolysis product of PGI2) and PGE₂ were performed as instructed by the manufacturer.

### EXAMPLE 5

### Biotin Switch Assay

S-nitrosylation of COX2 was determined by the biotin switch method as previously described (Atar et al., 2006). Briefly, myocardial samples from the left ventricular wall of rats treated with lipopolysaccharide (group 4) and pioglitazone+atorvastain (group 8) were homogenized in HEN buffer (25 mM HEPES (pH 7.7)-0.1 mM EDTA-0.01 mM necuproine). The supernatant containing membrane fragments and the cytosolic protein were recovered. The samples were incubated for 30 mins at 4° C. with blocking solution containing HEN buffer, 0.1% SDS and 20 mM N-ethylmaleimide (NEM) to block free thiols.

Lysates were centrifuged at 16,000×g for 10 min at 4° C. Cold acetone was added to precipitate the proteins. The pellets were resuspended in HEN buffer with 1% SDS, with 20 mM sodium ascorbate added to decompose the SNO bonds. The resulting free thiols in the sample were reacted with 0.05 mM biotinylating agent, biocytin (MPB) for 30 mins at room temperature. The excess MPB was removed by additional protein precipitation in cold acetone. COX2 was immunoprecipitated with anti-COX2 polyclonal antibody. Immunoprecipitates were washed three times with HEN buffer and resuspended in 50 μl of HEN containing Laemmli sample buffer, boiled at 95° C. for 5 min, loaded on 10% acrylamide gels and transferred to nitrocellulose. The biotinylated COX2 protein was detected with horseradish peroxidase-linked streptavidin. All procedures upto biotinylation were performed in the dark. The membranes were stripped with a stripping buffer and blotted again with anti-COX2 antibodies. The signal densities of the biotinylated COX2 and total COX2 were quantified by an image-scanning densitometer and the ratio of the densities was calculated for each animal.

### EXAMPLE 6

### Statistical Analysis

Data are expressed as mean ±SEM. Comparisons among the groups were performed by one-way ANOVA with Sidak correction for multiple comparisons (SPSS ver. 11.5.2.1). Values of P<0.05 were considered statistically significant.

### EXAMPLE 7

### The Effect of Aspirin on COX2 Induced by LPS

Aspirin alone (10 and 50 mg/kg) was observed to have no effect on myocardial 15-epi-LPXA4 levels (FIG. 2A). LPS increased myocardial 15-epi-LPXA4. Aspirin at 10 mg/kg did not significantly alter the effect of LPS on 15-epi-LPXA4 induction (p=0.08 versus LPS alone). However, aspirin at 50 mg/kg (p<0.001 versus LPS alone) and 200 mg/kg (p<0.001 versus LPS alone) augmented the LPS induction of 15-epi-LPXA4.

With regards to myocardial lipoxin A4, it was observed that Aspirin at 10 mg/kg and 50 mg/kg did not affect myocardial lipoxin A₄. LPS caused a small increase in myocardial lipoxin A₄ levels (FIG. 2B). Although Aspirin at 10 mg/kg and 50 mg/kg did not alter the LPS effect, it was observed that Aspirin at 200 mg/kg augmented the effect of LPS (p<0.001 versus each of the other groups).

Additionally, it was observed that Aspirin at 10 mg/kg did not affect myocardial 6-keto-PGF₁_{_{α}} levels (22.35 pg/mg versus 23.4310.24 pg/mg; p=0.48)(FIG. 2C). However, at 50 mg/kg, aspirin had a small but significant effect (20.97±0.51 pg/mg; p=0.001 versus the control group) on 6-keto-PGF₁_{_{—}} levels. LPS significantly increased myocardial 6-keto-PGF₁_{_{α}} levels (80.56±0.21 pg/mg; p<0.001 versus the control group). Aspirin at 10 mg/kg (76.70±0.25 pg/mg; p<0.001 versus LPS alone), 50 mg/kg (27.66±0.23 pg/mg; p<0.001 versus LPS alone), and 200 mg/kg (25.39±0.22 pg/mg; p<0.001 versus LPS alone) attenuated the effect of LPS in a dose-dependent manner.

Furthermore, it was observed that Aspirin at 10 mg/kg (34.48±0.21 pg/mg) and 50 mg/kg (33.68±0.24 pg/mg) did not significantly affect myocardial PGE₂ levels compared to the control group (36.98±0.37 pg/mg)(FIG. 2D). LPS caused a significant increase in myocardial PGE₂ levels (70.31±0.26; p<0.001 versus control). Aspirin at 10 mg/kg (48.70±3.02 pg/mg; p<0.001 versus LPS alone), 50 mg/kg (41.68±0.24 pg/mg; p<0.001 versus LPS alone), and 200 mg/kg (33.72±0.46 pg/mg; p<0.001 versus LPS alone) significantly attenuated the effect of LPS.

### EXAMPLE 8

### The Effect of Aspirin on COX2 Induced by Pioglitazone+Atorvastatin

The combined administration of Pioglitazone and atorvastatin significantly increased myocardial 15-epi-LPXA4 levels. In distinct contrast to the effect of aspirin on COX2 induced by LPS, aspirin at 10 mg/kg attenuated and at 50 mg/kg completely blocked the effect of pioglitazone and atorvastatin (FIG. 3A). In addition, 1400 W alone partially blocked the effect of pioglitazone plus atorvastatin. However, when both aspirin 50 mg/kg and 1400 W were administered to rats pretreated with pioglitazone plus atorvastatin, myocardial levels of 15-epi-LPXA4 increased to a greater extent than when pioglitazone+atorvastatin were administered alone.

Additionally, Pioglitazone+atorvastatin caused a small increase in myocardial lipoxin A₄ levels (FIG. 3B). This effect of pioglitazone+atorvastatin on myocardial lipoxin A₄ levels was not attenuated by either Aspirin and 1400 W alone or in combination. Furthermore, the combined administration of Pioglitazone+atorvastatin significantly increased myocardial 6-keto-PGF₁_{_{α}} levels (FIG. 3C) which was attenuated by 1400 W and aspirin at 10 mg/kg and 50 mg/kg. The combination of aspirin 50 mg/kg with 1400 W also partially blocked the effect of pioglitazone+atorvastatin.

With regards to myocardial PGE2 levels, combined administration of pioglitazone+atorvastatin significantly increased myocardial PGE₂ levels (FIG. 3D) which was attenuated by 10 mg/kg of aspirin. However, 50 mg/kg of aspirin, 1400 W and their combination completely blocked the effect of pioglitazone+atorvastatin.

### EXAMPLE 9

### S-Nitrosylation of COX2

In order to estimate the degree of S-nitrosylation of COX2, induced by either pioglitazone+atorvastatin or lipopolysaccharide, the biotin switch assay was used (FIG. 4). The assay showed a higher ratio of the signal density of the biotinylated COX2 to total COX2 in the pioglitazone+atorvastatin treated rats than in the lipopolysaccharide treated rats. This observation suggested that a larger percentage of the COX2 was S-nitrosylated in the pioglitazone+atorvastatin group than in the lipopolysaccharide group.

The following references were cited herein:
Atar S et al., Am J Physiol Heart Circ Physiol. 2006; 290:H1960-8.
Birnbaum Y et al., Cardiovasc Res. 2005; 65:345-355.
Birnbaum Y. Et al. Circulation. In press
Chiang N et al. J Pharmacol Exp Ther. 1998; 287:779-90.
Chiang N et al. Proc Natl Acad Sci USA. 2004; 101:15178-83.
Claria J and Serhan C N. Proc Natl Acad Sci USA. 1995; 92:9475-9.
Claria J and Planaguma A, Prostaglandins Leukot Essent Fatty Acids. 2005; 73:277-82.
Claria J et al., Mol. Med. 1996; 2:583-96.
Dalen J E. Am J. Med. 2006; 119:198-202.
Fiorucci S, et al. Faseb J. 2003; 17:1171-3.
Fiorucci S et al. Proc Natl Acad Sci USA. 2003; 100:10937-41.
Gilroy D W. Prostaglandins Leukot Essent Fatty Acids. 2005; 73:203-10.
Kim S F, et al., Science. 2005; 310:1966-70.
Patrono C et al., N Engl J. Med. 2005; 353:2373-83.
Paul-Clark M J et al. J Exp Med. 2004; 200:69-78.
Schneider C and Brash A R J Biol. Chem. 2000; 275:4743-6.
Serhan C N Prostaglandins Leukot Essent Fatty Acids. 2005; 73:141-62.
Serhan C N and Chiang N. Rheum Dis Clin North Am. 2004; 30:69-95.
Titos E et al., Am J. Physiol. 1999; 277:C870-7.
Van Wauwe J et al., Eicosanoids. 1991; 4:155-63.
Xu X M et al. Proc Natl Acad Sci USA. 1999; 96:5292-7.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of preventing cardiovascular events in an individual, comprising:
administering pharmacologically effective amounts of a composition comprising a non-aspirin selective COX1 inhibitor to the individual; and
administering pharmacologically effective amounts of a statin, a thiazolidinedione or a combination thereof, thereby preventing the cardiovascular events in the individual.

2. The method of claim 1, wherein the statin, the thiazolidinedione or the combination thereof is administered concurrent with, subsequent to or sequential to the administration of the non-aspirin selective COX1 inhibitor.

3. The method of claim 1, wherein the non-aspirin selective COX1 inhibitor inhibits the COX1 activity but does not have any effect on the COX2 activity.

4. The method of claim 1, wherein the non-aspirin selective COX1 inhibitor is SC560, FR122047, Resveratrol or Valeroyl Salicylate.

5. The method of claim 1, wherein the statin, the thiazolidinedione or the combination thereof activates COX2 via nitrosylation.

6. The method of claim 1, wherein the statin is atorvastatin, simvastatin, pravastatin, rosuvastatin, lovastatin or fluvastatin.

7. The method of claim 1, wherein the thiazolidinedione is pioglitazone or rosiglitazone.

8. The method of claim 1, wherein the individual is at risk of developing cardiovascular disease, has an established cardiovascular disease, is going to be subjected to cardiac surgery, non-cardiac surgery, percutaneous coronary interventions or a diabetic.

9. A method of preventing formation of blood clots in an individual receiving statin and/or thiazolidinedione, comprising the step of:
administering a pharmacologically effective amounts of a composition comprising a non-aspirin selective COX1 inhibitor to the individual.

10. The method of claim 9, wherein said administration of the non-aspirin selective COX1 inhibitor reduces the platelet activity and thereby prevents the formation of blood clots in the individual receiving statin and/or thiazolidinedione.

11. The method of claim 9, wherein the non-aspirin selective COX1 inhibitor has no effect on the activity of COX2.

12. The method of claim 9, wherein the non-aspirin selective COX1 inhibitor is SC560, FR122047, Resveratrol or Valeroyl Salicylate.

13. The method of claim 9, wherein the statin and/or the thiazolidinedione is administered concurrent with, subsequent to or sequential to the administration of the non-aspirin selective COX1 inhibitor.

14. The method of claim 13, wherein the statin and/or the thiazolidinedione activates COX2 via nitrosylation.

15. The method of claim 14, wherein the statin is atorvastatin simvastatin, pravastatin, rosuvastatin, lovastatin or fluvastatin.

16. The method of claim 14, wherein the thiazolidinedione is pioglitazone or rosiglitazone.

17. The method of claim 9, wherein the individual is at risk of developing cardiovascular disease, has an established cardiovascular disease, is going to be subjected to cardiac surgery, non-cardiac surgery, percutaneous coronary interventions or a diabetic.

18. A method of treating an individual at risk for or with an established cardiovascular disease, comprising:
administering pharmacologically effective amounts of non-aspirin selective COX1 inhibitor; and
administering pharmacologically effective amounts of atorvastatin, pioglitazone or a combination thereof, thereby treating the individual at risk for or with the established cardiovascular disease.

19. The method of claim 18, wherein the atorvastatin, the pioglitazone or the combination thereof is administered concurrent with, subsequent to or sequential to the administration of the non-aspirin selective COX1 inhibitor.

20. The method of claim 18, wherein the non-aspirin selective COX1 inhibitor blocks the COX1 activity but does not interfere with the inhibition of COX2 activity by the atorvastatin, the pioglitazone or the combination thereof.
